(19) United States
(12) Patent Application Publication
(10) Pub. No.: US 2024/0238587 A1
(43) Pub. Date: Jul. 18, 2024

(54) IMPLANTABLE MEDICAL LEAD AND RELATED DEVICES AND METHODS

(71) Applicant: Foresite Capital Holdings, LLC, St. George, UT (US)

(72) Inventors: Jon Obray, St. George, UT (US); Rick Obray, St. George, UT (US); Eric Olson, St. George, UT (US)

(21) Appl. No.: 18/559,238

(22) PCT Filed: May 6, 2022
(86) PCT No.: PCT/US22/28089

§ 371 Date: Nov. 6, 2023

Related U.S. Application Data
(60) Provisional application No. 63/185,166, filed on May 6, 2021.

(51) Int. Cl. A61N 1/05

(52) U.S. Cl. CPC A61N 1/0553; A61N 1/0558

(57) ABSTRACT

An implantable medical lead for electrical stimulation may include a body, which may include a flexible distal end, a flexible proximal end, and a rigid portion extending between the flexible distal end and the flexible proximal end. The body may include an aperture extending through the flexible distal end and the flexible proximal end and configured to receive a guidewire therethrough. The implantable medical lead may include an electrode coupled to the rigid portion and an electrical conductor electrically connected to the electrode and extending from the electrode through the flexible proximal end. A medical anchor to secure the implantable medical lead may include a body and a clamp element configured to move between an open position and a closed position. A surgical instrument to implant the implantable medical lead may include a first arm having a first clamp surface and a second arm having a second clamp surface.

background is only provided to illustrate one example technology area where some implementations described herein may be practiced.

### SUMMARY

The present disclosure relates generally to an implantable medical lead for electrical stimulation, as well as related devices and methods. Various features and aspects disclosed herein provide a unique implantable medical lead compatible for use with tubes and open spinal procedures, thereby allowing clinicians to accurately and safely implant the medical lead via minimally invasive surgical procedures incorporating only a mini-incision and partial laminectomy.

In some embodiments, an implantable medical lead for electrical stimulation may include a body, which may include a flexible distal end and/or a flexible proximal end. In some embodiments, the implantable medical lead may include a rigid portion, which may extend between the flexible distal end and the flexible proximal end.

In some embodiments, the body may include an aperture extending through the flexible distal end and the flexible proximal end and configured to receive a guidewire therethrough. In some embodiments, the implantable medical lead may include an electrode coupled to the rigid portion. In some embodiments, the implantable medical lead may include an electrical conductor electrically connected to the electrode and extending from the electrode through the flexible proximal end.

In some embodiments, a top surface of the flexible distal end may be tapered downwardly in a distal direction. In some embodiments, a cross-section of the body may be dome-shaped or triangular. In some embodiments, the electrode may be disposed on a bottom surface of the body. In some embodiments, the flexible proximal end may be tapered inwardly such that a width of the body decreases in a proximal direction. In some embodiments, the bottom surface of the body may be flat.

In some embodiments, the body may include one or more circular radiopaque markers.

In some embodiments, the circular radiopaque markers may include a first circular radiopaque marker and a second circular radiopaque marker disposed within the flexible proximal end. In some embodiments, the first circular radiopaque marker and the second circular radiopaque marker may be equidistant from a longitudinal axis of the body and aligned. In some embodiments, the body may include one or more holes, and each of the holes may include a circular radiopaque marker.

In some embodiments, the implantable medical lead may include another electrode coupled to the rigid portion and another electrical conductor electrically connected to the other electrode and extending from the other electrode through the flexible proximal end. In some embodiments, the flexible proximal end may include a first opening and a second opening. In some embodiments, the electrical conductor may extend through the first opening and the other electrical conductor may extend through the second opening. In some embodiments, the aperture may be disposed above and in between the first opening and the second opening.

In some embodiments, the implantable medical lead may include multiple electrodes arranged in a first line and multiple other electrodes arranged in a second line parallel to the first line. In some embodiments, the multiple electrodes may include the electrode, and the multiple other electrodes may include the other electrode. In some embodiments, the electrical conductor may be electrically connected to the multiple electrodes, and the other electrical conductor may be electrically connected to the multiple other electrodes.

In some embodiments, a medical anchor to secure the implantable medical lead may include a body, which may include one or more of the following: a distal end, a proximal end, an opening extending through the distal end, a groove extending through the proximal end and proximate the opening, a first suture hole, and a second suture hole. In some embodiments, the first suture hole may oppose the second suture hole. In some embodiments, the opening and the groove may be configured to receive the electrical conductor.

In some embodiments, the medical anchor may include a clamp element, which may be hinged to the body of the medical anchor. In some embodiments, the clamp element may be configured to move between an open position and a closed position. In some embodiments, an inner surface of clamp element may include another groove configured to align with the groove when the clamp element is in the closed position. In some embodiments, the clamp element may be configured to clamp the electrical conductor between the clamp element and the body of the medical anchor when the clamp element is in the closed position to prevent the electrical conductor from sliding through the medical anchor.

In some embodiments, the first suture hole and the second suture hole may be disposed within the distal end and extend through a top surface of the body of the medical anchor and a bottom surface of the body of the medical anchor. In some embodiments, the body of the medical anchor may include a cutout portion extending between a first wall and a second wall. In some embodiments, the first wall and the second wall may be configured to contact a first side and a second side, respectively, of the clamp element when the clamp element is in the closed position. In some embodiments, the first wall and the second wall may be spaced apart by a floor of the body of the medical anchor. In some embodiments, the groove may extend through the floor. In some embodiments, the inner surface of the clamp element may be configured to contact the floor when the clamp element is in the closed position.

In some embodiments, a surgical instrument to implant the implantable medical lead for electrical stimulation. In some embodiments, a first arm may include a first distal end, which may include a first clamp surface. In some embodiments, the first clamp surface may include a circular peg configured to insert into a circular hole of an implantable medical lead. In some embodiments, the surgical instrument may include an aperture extending through the first arm proximal to the first distal end. In some embodiments, the aperture may be configured to receive one or more electrical conductors and a guidewire therethrough.

In some embodiments, the surgical instrument may include a second arm coupled to the first arm. In some embodiments, the second arm may include a second distal end. In some embodiments, the second distal end may include a second clamp surface opposing the first clamp surface. In some embodiments, the second clamp surface may include another circular peg configured to insert into another circular hole of the implantable medical lead. In some embodiments, the second arm may include a groove opposing the aperture.

In some embodiments, the first clamp surface may include a first rounded indent. In some embodiments, the second clamp surface may include a second rounded indent. In some embodiments, the first peg and the second peg may extend from the first rounded indent and the second rounded indent, respectively. In some embodiments, the surgical instrument may be scissor-tong shaped.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example implantable medical lead, according to some embodiments;

FIG. 1B is a top view of the implantable medical lead, according to some embodiments;

FIG. 1C is a bottom view of the implantable medical lead, according to some embodiments;

FIG. 1D is a side view of the implantable medical lead, according to some embodiments;

FIG. 1E is a proximal end view of the implantable medical lead, according to some embodiments;

FIG. 1F is a distal end view of the implantable medical lead, according to some embodiments;

FIG. 1G is an upper perspective view of the implantable medical lead, illustrating an example flexible proximal end in a first position, according to some embodiments;

FIG. 1H is an upper perspective view of the implantable medical lead, illustrating the flexible proximal end in a second position, according to some embodiments;

FIG. 1I is an upper perspective view of an example flexible distal end, according to some embodiments;

FIG. 1J is a lower perspective view of the implantable medical lead, illustrating the implantable medical lead just prior to clamping with an example surgical instrument, according to some embodiments;

FIG. 2A is an upper perspective view of an example cannula, illustrating the implantable medical lead and an example guidewire disposed within the cannula, according to some embodiments;

FIG. 2B is a proximal end view of the cannula, illustrating the implantable medical lead and the guidewire disposed within the cannula, according to some embodiments;

FIG. 2C is a side view of the cannula, illustrating the guidewire extending therethrough, according to some embodiments;

FIG. 3A is an upper perspective view of the surgical instrument, according to some embodiments;

FIG. 3B is an enlarged upper perspective view of a distal end of the surgical instrument, according to some embodiments;

FIG. 3C is an enlarged lower perspective view of the distal end of the surgical instrument, according to some embodiments;

FIG. 3D is an enlarged lower perspective view of the distal end of the surgical instrument, according to some embodiments;

FIG. 3E is an upper perspective of the surgical instrument disposed within the cannula, according to some embodiments;

FIG. 3F is an enlarged upper perspective view of the surgical instrument disposed within the cannula, according to some embodiments;

FIG. 4A is an upper perspective view of an example medical anchor, illustrating an example clamp element in an open position, according to some embodiments;

FIG. 4B is an upper perspective view of the medical anchor, illustrating the clamp element in a closed position, according to some embodiments;

FIG. 4C is an upper perspective view of an example body of the medical anchor, according to some embodiments;

FIG. 5A is a cross-sectional view of the cannula, illustrating the implantable medical lead, the guidewire, and the medical anchor inside the cannula, according to some embodiments;

FIG. 5B is a lower perspective view of the cannula, illustrating the implantable medical lead, the guidewire, and the medical anchor inside the cannula, according to some embodiments;

FIG. 6A is an upper perspective view of another example medical anchor, illustrating the medical anchor in an open position, according to some embodiments;

FIG. 6B is another upper perspective view of the medical anchor of FIG. 6A, illustrating the medical anchor in the open position, according to some embodiments;

FIG. 6C is a lower perspective view of the medical anchor of FIG. 6A, illustrating the medical anchor in the open position, according to some embodiments;

FIG. 6D is a top view of the medical anchor of FIG. 6A, illustrating the medical anchor in the open position, according to some embodiments;

FIG. 6E is another upper perspective view of the medical anchor of FIG. 6A, illustrating the medical anchor in an example closed position, according to some embodiments;

FIG. 6F is another upper perspective view of the medical anchor of FIG. 6A and the implantable medical lead extending therethrough, illustrating the medical anchor in an example closed position, according to some embodiments;

FIG. 6G is an upper perspective view of the medical anchor of FIG. 6A and example electrical conductors extending therethrough, illustrating the medical anchor the open position, according to some embodiments;

FIG. 6H is an upper perspective view of the medical anchor of FIG. 6A and the electrical conductors extending therethrough, illustrating the medical anchor the closed position, according to some embodiments;

FIG. 6H is another upper perspective view of the medical anchor of FIG. 6A and the electrical conductors extending therethrough, illustrating the medical anchor the closed position, according to some embodiments;

FIG. 7A illustrates another example cannula inserted into an epidural space, according to some embodiments;

FIG. 7B illustrates the guidewire inserted through the other cannula, according to some embodiments;

FIG. 7C illustrates an example inner cannula being removed from the other cannula, according to some embodiments;

FIG. 7D illustrates another example implantable medical lead being implanted through the cannula, according to some embodiments;

FIG. 8A illustrates the other implantable medical lead coupled to an example implantable pulse generator, according to some embodiments; and

FIG. 8B illustrates the other implantable medical lead coupled to an example radiofrequency receiver, according to some embodiments.

### DESCRIPTION OF EMBODIMENTS

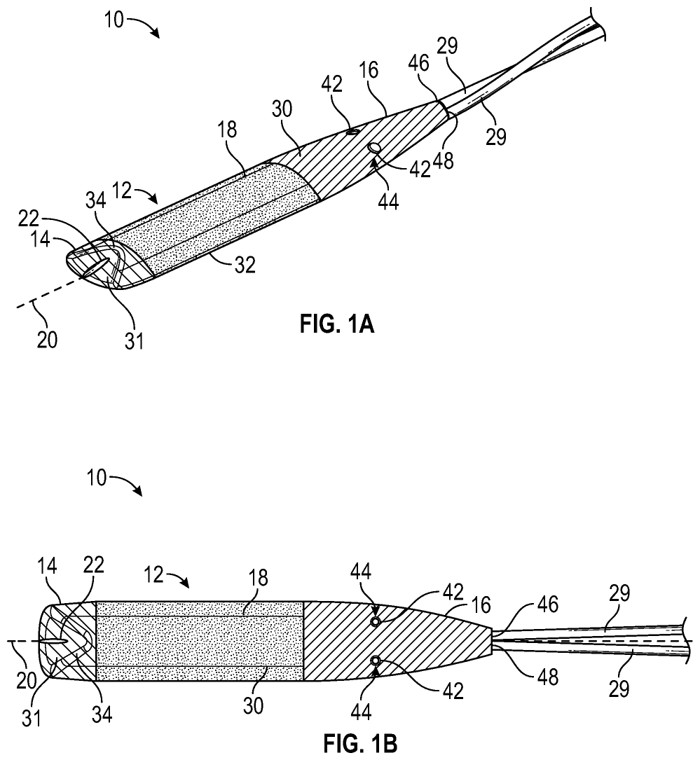

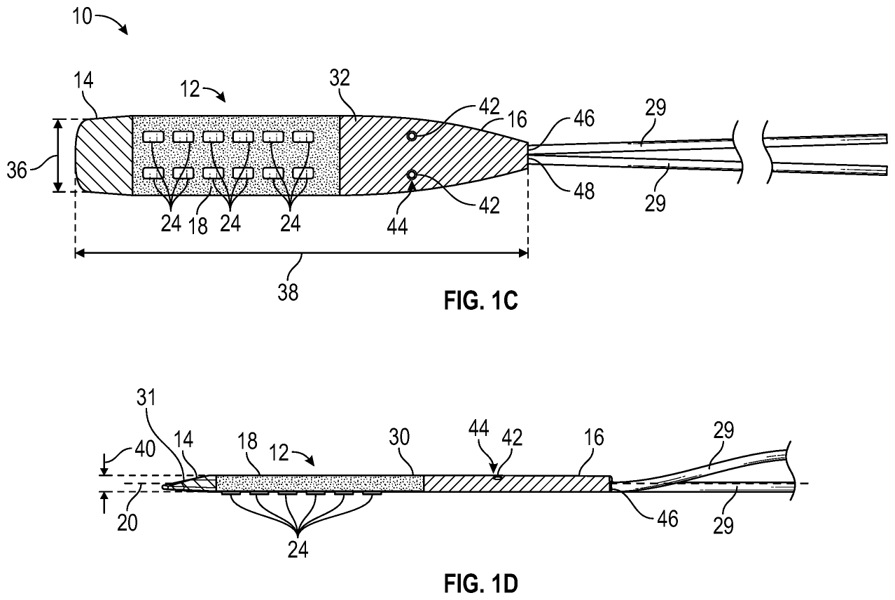

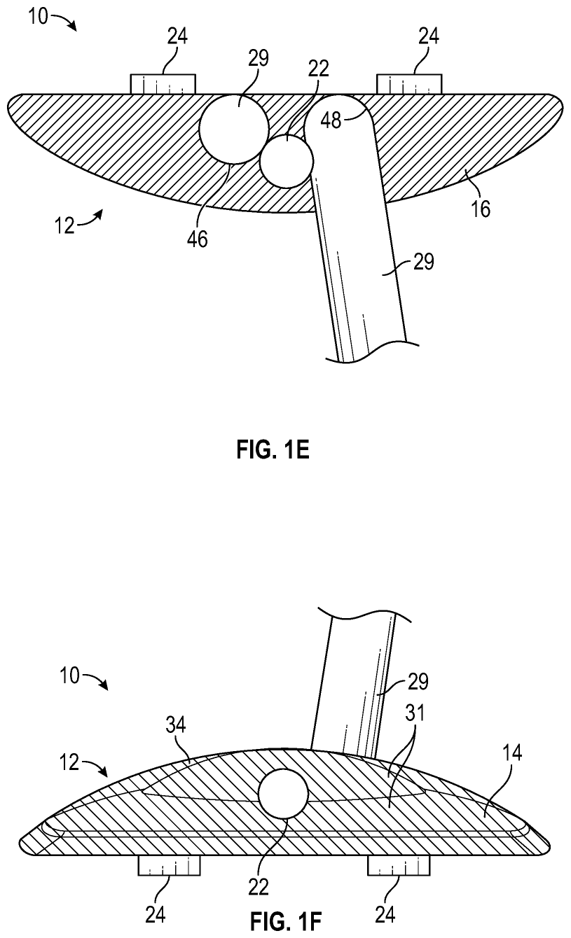

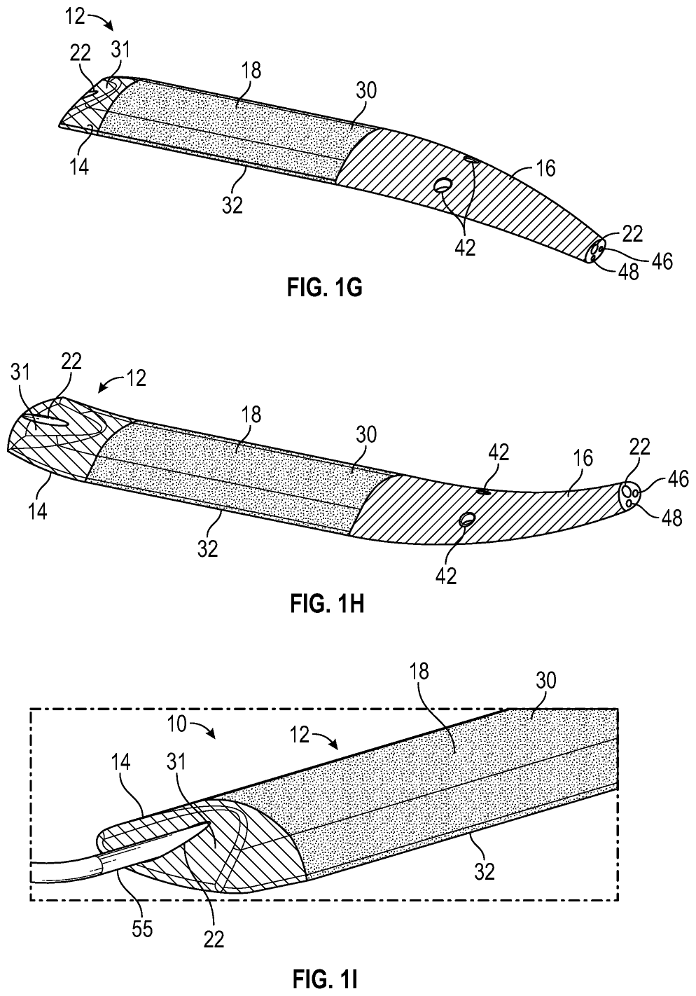

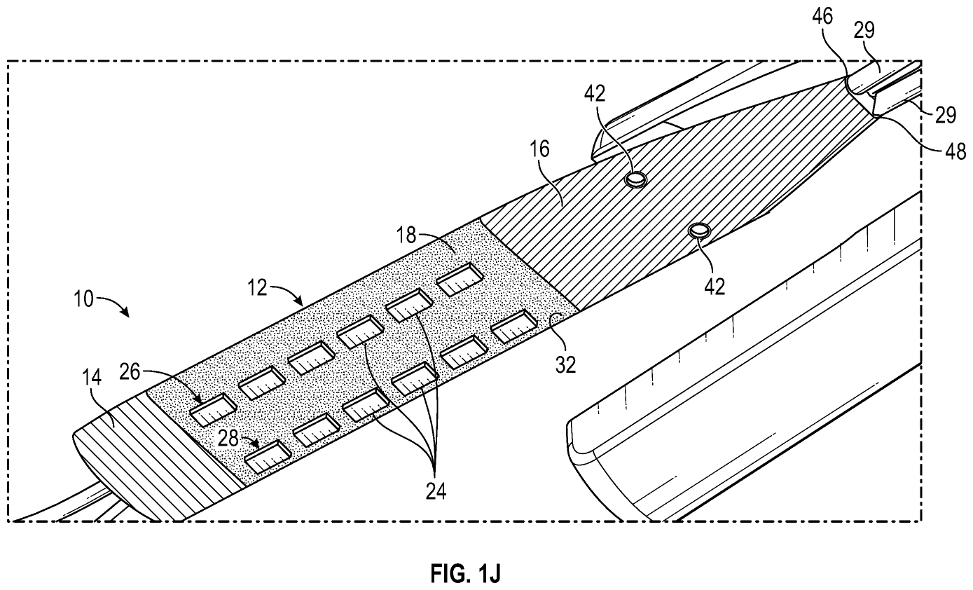

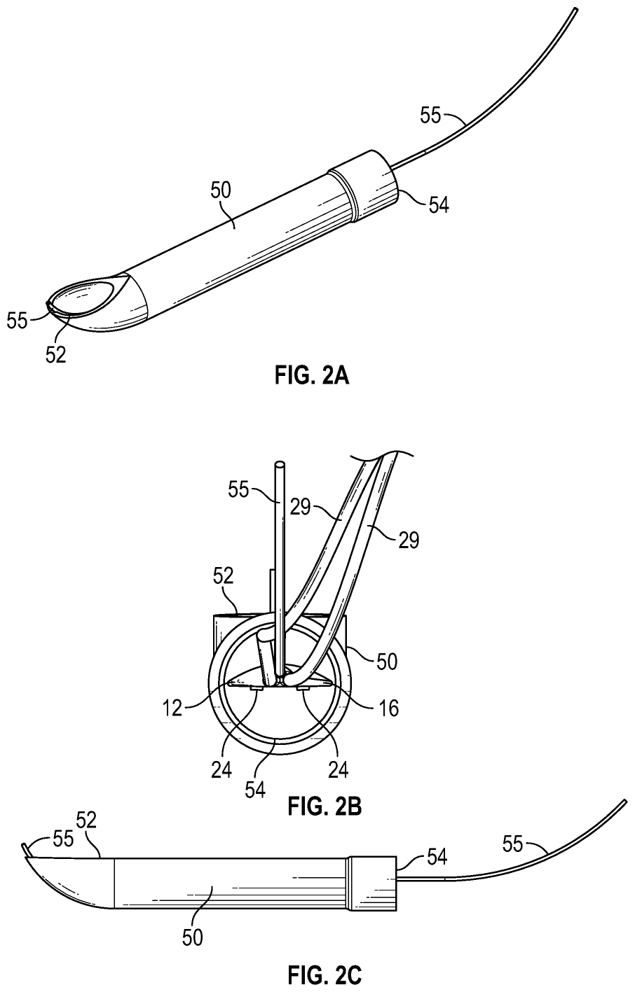

Referring now to FIGS. 1A-1J, an implantable medical lead 10 for electrical stimulation is illustrated, according to some embodiments. In some embodiments, the implantable medical lead 10 may include a body 12, which may include a flexible distal end 14 and/or a flexible proximal end 16. In some embodiments, the flexible distal end 14 and/or the flexible proximal end 16 may reduce a risk of trauma or damaging the spinal cord during insertion into the epidural space. In further detail, the flexible distal end 14 and/or the flexible proximal end 16 may be configured to bend and make the turn from a cannula into foramen of the spine without damaging delicate structures near the spinal cord. The cannula is described further with respect to FIGS. 2A-2C, according to some embodiments.

In some embodiments, the implantable medical lead 10 may include a rigid portion 18, which may extend between the flexible distal end 14 and the flexible proximal end 16. The rigid portion 18 is illustrated by a first shading in FIGS. 1A-1J, the flexible distal end 14 is illustrated by a second shading in FIGS. 1A-1J, and the flexible proximal end 16 is illustrated by a third shading in FIGS. 1A-1J, according to some embodiments. In some embodiments, the rigid portion 18 may have a longitudinal axis 20. As used in the present disclosure, the term “distal” refers to a portion of a device further from the clinician, and the term “proximal” refers to a portion of a device closer to the clinician.

In some embodiments, the flexible distal end 14 and/or the flexible proximal end 16 may be constructed of plastic, an elastomer, or another suitable material. In these and other embodiments, the flexible proximal end 16 may be constructed of a material that allows the flexible proximal end 16 to bend or flex from a first position, illustrated in FIG. 1G, to a second position, illustrated in FIG. 1H. In some embodiments, the first position may correspond to an angle between about 30° and about 45° below the longitudinal axis 20. In some embodiments, the second position may correspond to an angle between about 30° and about 45° above the longitudinal axis 20, which may facilitate insertion of the body 12 through the cannula and into the foramen of the spine.

In some embodiments, the flexible distal end 14 may be constructed of a same or different material than the flexible proximal end 16 that allows the flexible distal end 14 to bend from a first position to a second position. In some embodiments, the first position may correspond to an angle between about 30° and about 45° below the longitudinal axis 20. In some embodiments, the second position may correspond to an angle between about 30° and about 45° above the longitudinal axis 20, which may facilitate insertion of the body 12 through the cannula and into the foramen of the spine. In some embodiments, the body 12 may be generally flat or aligned with the longitudinal axis 20 along an entire length of the body 12 when the body 12 is in a resting or unbiased position without application of force, as illustrated, for example, in FIG. 1D.

In some embodiments, the rigid portion 18 may be constructed of plastic, metal, or another suitable material. In some embodiments, the rigid portion 18 may be constructed of a plastic with a greater durometer than a plastic of the flexible distal end 14 and/or the flexible proximal end 16. In some embodiments, the body 12 may include an aperture 22 extending through the flexible distal end 14 and the flexible proximal end 16 and configured to receive a guidewire therethrough. In some embodiments, the aperture 22 may extend longitudinally through the body 12.

In some embodiments, the implantable medical lead 10 may include one or more electrodes 24, which may be coupled to the rigid portion 18 such that the electrodes 24 are conductively exposed. In some embodiments, some of the electrodes 24 may be disposed in a first line 26 and some of the electrodes 24 may be disposed in a second line 28 parallel to the first line 26, which may facilitate. In some embodiments, the electrodes 24 may be coupled to the rigid portion 18 in any suitable arrangement or pattern.

In some embodiments, one or more electrical conductors 29 may be electrically connected to the electrodes 32. In some embodiments, the electrical conductors 29 may extend from the electrodes 32 through the flexible proximal end 16. In some embodiments, a particular electrical conductor 29 may be electrically coupled to one or more particular electrodes 32 in the first line 26, and another particular electrical conductor 29 may be electrically coupled to one or more other particular electrodes 32 in the second line 28.

In some embodiments, a shape of the body 12 may be configured to facilitate insertion of the implantable medical lead 10 into the epidural space. In some embodiments, the shape of the body 12 may also increase contact of the electrodes 32 with a targeted tissue, while reducing a risk of developing scar tissue on the body 12. In further detail, in some embodiments, a top surface 30 of the flexible distal end 14 may be tapered downwardly in a distal direction, as illustrated, for example, in FIGS. 1F and 1I, which may facilitate insertion of the implantable medical lead 10 into the epidural space. In some embodiments, a proximal end of a tapered surface 31 that is tapered downwardly in the distal direction may include a rounded edge 34, which may facilitate smooth insertion. In some embodiments, the flexible distal end 14 may be beveled or chamfered.

In some embodiments, the tapered surface 31 may extend near or to a distal-most surface of the flexible distal end 14. In some embodiments, the tapered surface 31 may be planar and tapered downwardly in the distal direction at a single angle. In some embodiments, the tapered surface 31 may be tapered downwardly in the distal direction at more than one angle, as illustrated, for example in FIG. 1F, having a steeper proximal portion compared to a distal portion of the tapered surface 31, for example. In some embodiments, the aperture 22 may be disposed within the tapered surface 31.

In some embodiments, the electrodes 24 may be disposed on a bottom surface 32 of the body 12. In some embodiments, when the implantable medical lead 10 is inserted into the epidural space, the top surface 30 may be posterior to the bottom surface 32. In some embodiments, the bottom surface 32 may be anterior to the top surface 30 when the implantable medical lead 10 is inserted into the epidural space. As used in the present disclosure, the term “above” may refer to a posterior direction when the implantable medical lead 10 is inserted into the epidural space, and the term “below” may refer to an anterior direction when the implantable medical lead 10 is inserted into the epidural space. In some embodiments, the bottom surface 32 of the body 12 may be flat, which may facilitate contact of the electrodes 24 on the bottom surface 32 with the targeted tissue. In some embodiments, the electrodes 24 may be located on a portion of the body 12 other than the bottom surface 32.

As illustrated, for example, in FIGS. 1C and 1J, the flexible proximal end 16 may be tapered inwardly such that a width 36 of the body 12 decreases in a proximal direction, which may decrease a size of the body 12 and increase maneuverability within the epidural space. FIG. 1C illustrates a length 38 of the body 12 and the width 36 of the body 12, according to some embodiments. In some embodiments, a height 40 of the body 12, illustrated, for example, in FIG. 1D, may be for application to a human spinal cord. In some embodiments, the body 12 may be configured to be applied to cervical vertebrae in the human spinal cord, thoracic vertebrae in the human spinal cord, or lumbar vertebrae in the human spinal cord.

In some embodiments, the body 12 may include one or more radiopaque markers 42, which may be circular. In some embodiments, a clinician may determine that the implantable medical lead 10 is inserted within the epidural space in a straight manner by observing the radiopaque markers 42 that are circular as perfect circles. In some embodiments, the radiopaque markers 42 may include a first radiopaque marker 42a and a second radiopaque marker 42b, which may be disposed within the flexible proximal end 16. In some embodiments, the first radiopaque marker 42a and the second radiopaque marker 42b may be equidistant from a center or a longitudinal axis of the body 12 and aligned with each other, which may provide a clear indication of a position of the implantable medical lead 10 to the clinician, who may observe the first radiopaque marker 42a and the second radiopaque marker 42b as perfect circles when the implantable medical lead 10 is inserted correctly.

In some embodiments, the body 12 may include one or more holes 44 configured to receive one or more pegs of a surgical instrument, which the clinician may use to insert the implantable medical lead 10. In some embodiments, each of the holes 44 may be circular and constructed of a radiopaque material. In further detail, in some embodiments, a circular bottom of each of the holes 44 may be constructed of the radiopaque material and/or a circular edge of each of the holes 44 may be constructed of the radiopaque material. Thus, the holes 44 may each serve as the radiopaque markers 42.

In some embodiments, the flexible proximal end 16 may include a first opening 46 and/or a second opening 48. In some embodiments, a particular electrical conductor 29 may extend through the first opening 46 and/or another particular electrical conductor 29 may extend through the second opening 48. In some embodiments, the aperture 22 may be disposed above and in between the first opening 46 and the second opening 48, which may accommodate a dome or triangle shape of the body 12 to improve insertion of the body 12 into the epidural space. In some embodiments, the first opening 46, the second opening, and the aperture 22 may be disposed on a proximal face or proximal-most surface of the flexible proximal end 16.

Referring now to FIGS. 2A-2C, in some embodiments, a cannula 50 may be inserted by the clinician through the skin of the patient and into the epidural space, creating a small incision. In some embodiments, the implantable medical lead 10 may be configured to fit in and move through the cannula 50 into the epidural space. In some embodiments, in order to insert the cannula 50 and place the implantable medical lead 10 in the epidural space, only a small portion of the lamina may be removed, or no lamina may be removed at all. This is in contrast to a typical laminotomy used to insert a medical lead having a flat, rectangular paddle shape, which results in a fairly large incision.

As mentioned, a full laminectomy involves a large resection and removal of vertebral bone and tissue. Because the surgical lead is surgically implanted in this manner, a trained Interventional Pain Management physician may not perform the procedure. The unique design features of the lead allow for the lead to be placed with any minimally invasive techniques that allows for a limited laminotomy for epidural space access, where the lead can be guided with fluoroscopy over a guide wire and with the steering tools. For example, the implantable medical lead 10 may be inserted into the epidural space using a surgical procedure, which may be minimally invasive. In some embodiments, the implantable medical lead 10 may be inserted into the epidural space by a surgeon trained in minimally invasive surgical procedures. In some embodiments, during the minimally invasive surgical procedure, an endoscope or tube may be used to reach the epidural space through a very small incision, and the guidewire 55 and/or the radiopaque markers 42 may facilitate proper placement of the implantable medical lead 10 without an “open” surgical procedure. In some embodiments, the cannula 50 may correspond to a cannula of the endoscope or tube. In some embodiments, the implantable medical lead 10 may correspond to a cannulated electrical stimulation lead.

In some embodiments, a distal opening 52 of the cannula 50 may be generally perpendicular to a proximal opening 54 of the cannula 50, which may facilitate guidance of the implantable medical lead 10 into the epidural space. A guidewire 55 is illustrated extending through the aperture 22, according to some embodiments.

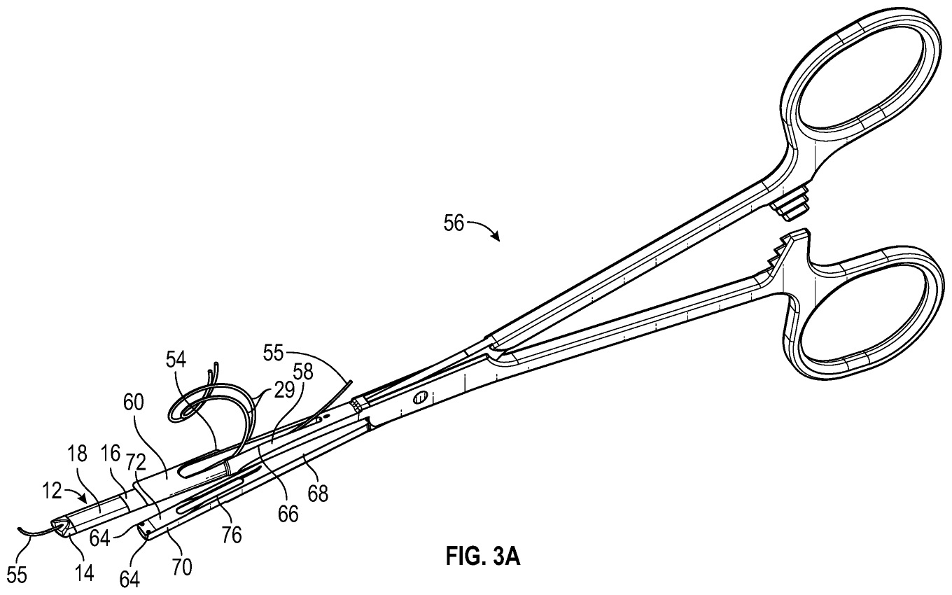

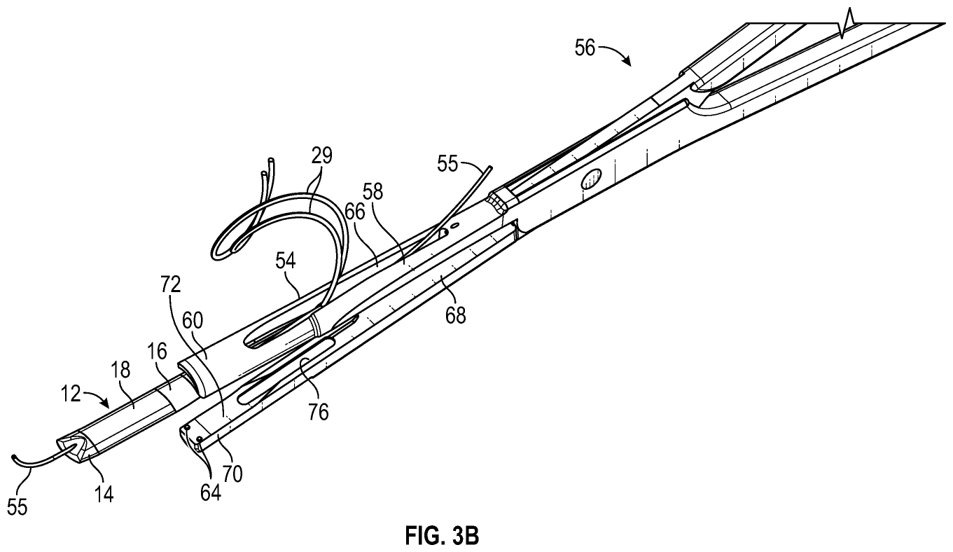

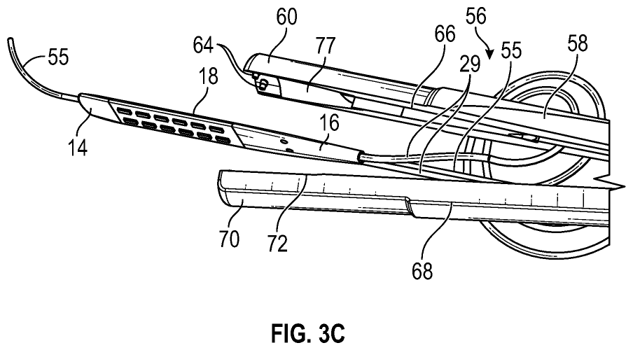

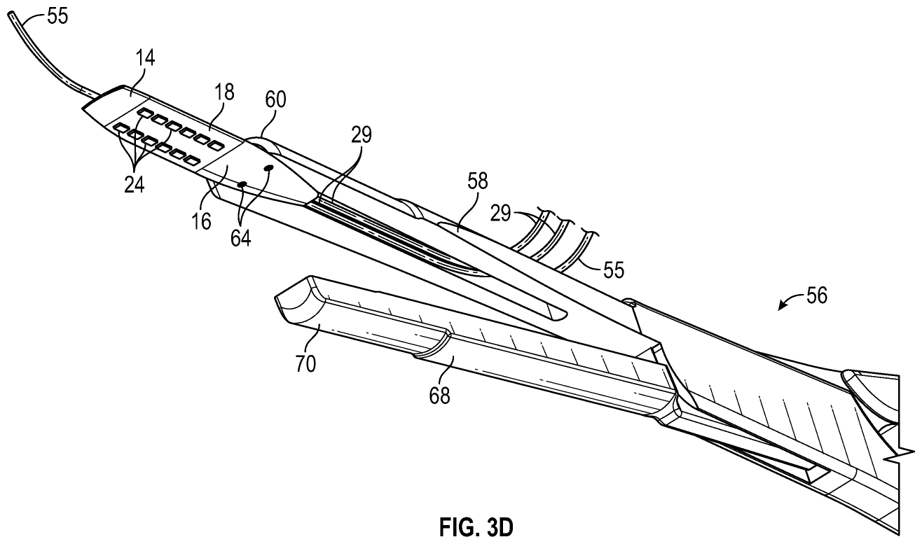

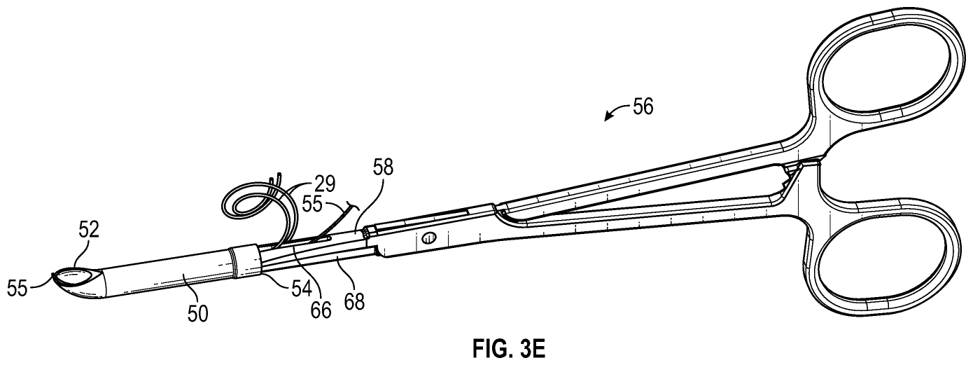

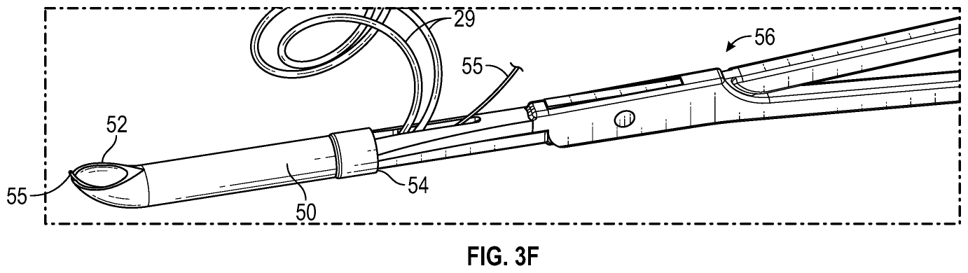

Referring now to FIGS. 3A-3F, a surgical instrument 56 to implant the implantable medical lead 10 for electrical stimulation is illustrated, according to some embodiments. In some embodiments, a first arm 58 of the surgical instrument 56 may include a first distal end 60, which may include a first clamp surface 62. In some embodiments, the first clamp surface 62 may include one or more pegs 64 each configured to insert into a particular hole 44 of the implantable medical lead 10. In some embodiments, the pegs 64 may be circular, and the particular hole 44 may be circular.

In some embodiments, the surgical instrument 56 may include an aperture 66 extending through the first arm 58 proximal to the first distal end 60 and which may be elongated. In some embodiments, the aperture 66 may be configured to receive one or more electrical conductors 29 and the guidewire 55 therethrough. In some embodiments, the aperture 66 may be elongated, and the electrical conductors 29 and/or the guidewire 55 may be loosely disposed within the aperture 66, which may allow the surgical instrument 56 to move independently of the electrical conductors 29 and/or the guidewire 55.

In some embodiments, the surgical instrument 56 may include a second arm 68 coupled to the first arm 58. In some embodiments, the second arm 68 may include a second distal end 70. In some embodiments, the second distal end 70 may include a second clamp surface 72 opposing the first clamp surface 62. In some embodiments, the surgical instrument 56 may be configured to clamp the body 12 between the first clamp surface 62 and the second clamp surface 72. In some embodiments, the second clamp surface 72 may include one or more of the pegs 64, which may be configured to insert into another of the holes 44 of the implantable medical lead 10. In some embodiments, the pegs 64 and the other of the holes 44 may be circular. In some embodiments, the second arm 68 may include a groove 76 opposing the aperture 66, and the groove 76 may provide space for the electrical conductors 29 and/or the guidewire 55.

In some embodiments, the first clamp surface 62 may include a rounded indent 77 and/or the second clamp surface 72 may include flat or planar surface. Thus, in some embodiments, the rounded indent 77 may be configured to receive the body 12 of the implantable medical lead 10 when the surgical instrument 56 clamps the body 12, which may be rounded. In some embodiments, one or more particular pegs 64 may extend from the first rounded indent 77 and one or more other particular pegs 64 may extend from the second clamp surface, which may be flat.

In some embodiments, the surgical instrument 56 may be scissor-tong shaped, with the first arm 58 and the second arm 68 pivotally coupled to each other. In some embodiments, the first arm 58 and the second arm 68 may be configured to fit within the cannula 50, and the surgical instrument 56 may be configured to insert the implantable medical lead 10 into the patient through the cannula 50.

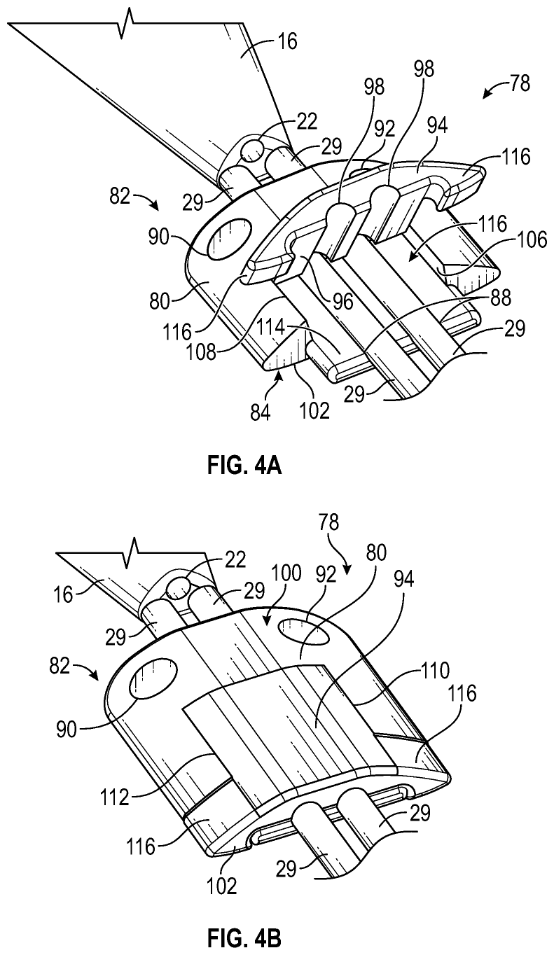

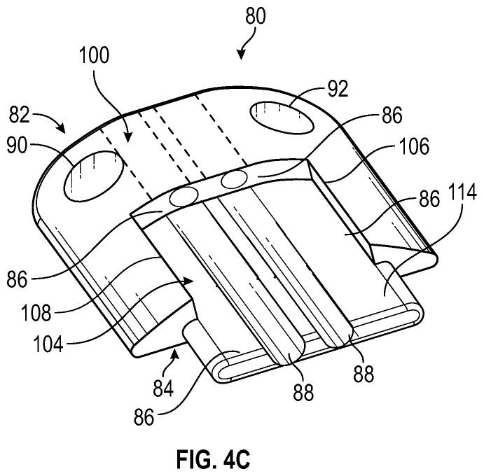

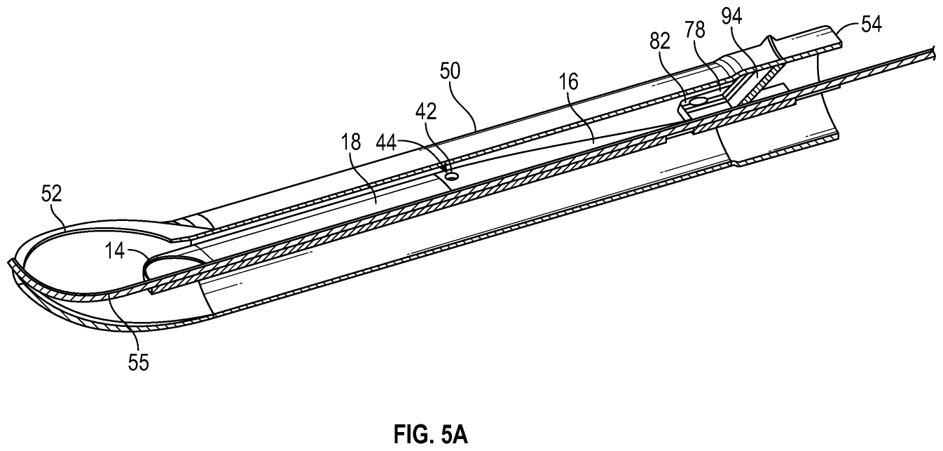

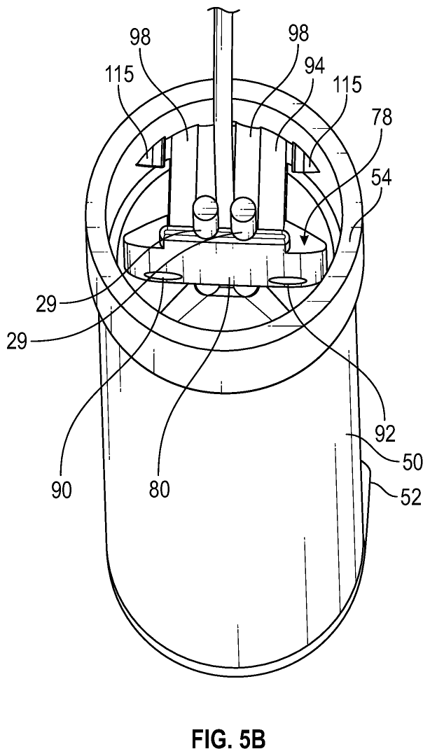

Referring now to FIGS. 4A-5D, a medical anchor 78 to secure the implantable medical lead 10 within a body of the patient is illustrated, according to some embodiments. In some embodiments, the medical anchor 78 may include a body 80, which may include one or more of the following: a distal end 82; a proximal end 84; one or more openings 86 extending through the distal end 82; one or more grooves 88 extending through the proximal end 84 and proximate the openings 86; a first suture hole 90; and a second suture hole 92. In some embodiments, the first suture hole 90 may oppose the second suture hole 92 to provide securement of the medical anchor 78 on both sides of the body 80. In some embodiments, suture may be inserted through the first suture hole 90 and/or the second suture hole 92 and secured to tissue of the patient to prevent migration of the implantable medical lead 10 after implantation. In some embodiments, each of the openings 86 and each of the grooves 88 may be configured to receive a particular electrical conductor 29. In some embodiments, each of the openings 36 may include a tunnel.

In some embodiments, the medical anchor 78 may include a clamp element 94, which may be hinged to the body 80 at a distal end of the clamp element 94. In some embodiments, the clamp element 94 may be configured to move between an open position, illustrated, for example, in FIG. 4A, and a closed position, illustrated, for example, in FIG. 4B. In some embodiments, an inner surface 96 of the clamp element 94 may include one or more other grooves 98 configured to align with the grooves 88 when the clamp element 94 is in the closed position. In some embodiments, the clamp element 94 may be configured to clamp the electrical conductors 29 between the clamp element 94 and the body 80 when the clamp element 94 is in the closed position to prevent the electrical conductors 29 from sliding through the medical anchor 78. In some embodiments, the clamp element 94 may be moved to the closed position after the implantable medical lead 10 is in a desired position.

In some embodiments, the first suture hole 90 and the second suture hole 92 may be disposed within the distal end 82 and extend through a top surface 100 of the body 80 and a bottom surface 102 of the body 80. In some embodiments, the body 80 may include a cutout portion 104 extending between a first wall 106 and a second wall 108. In some embodiments, the first wall 106 and the second wall 108 may be configured to contact a first side 110 and a second side 112, respectively, of the clamp element 94 when the clamp element 94 is in the closed position. In some embodiments, the first wall 106 and the second wall 108 may be spaced apart by a floor 114 of the body 80. In some embodiments, the grooves 88 may extend through the floor 114. In some embodiments, the inner surface 96 of the clamp element 94 may be configured to contact the floor 114 when the clamp element 94 is in the closed position, which may prevent fluid from entering the implantable medical lead 10.

In some embodiments, the floor 114 may extend proximal to the first wall 106 and the second wall 108. In some embodiments, the clamp element 94 may include opposing arms 115, which may be configured to contact an edge of the body 80 proximate the floor 114 when the clamp element 94 is in the closed position. In some embodiments, a top surface of the body 80 and/or a top surface of the clamp element 94 may be rounded or dome-shaped to improve insertion of the medical anchor 78 into the epidural space. In some embodiments, a bottom of the body 80 may be flat. In some embodiments, when the clamp element 94 is in the closed position, the top surface of the clamp element 94 may be aligned with the top surface of the body 80 and/or a bottom surface of the clamp element 94 may be aligned with a bottom surface of the body 80 such that the medical anchor is smooth and does not irritate or injure the patient.

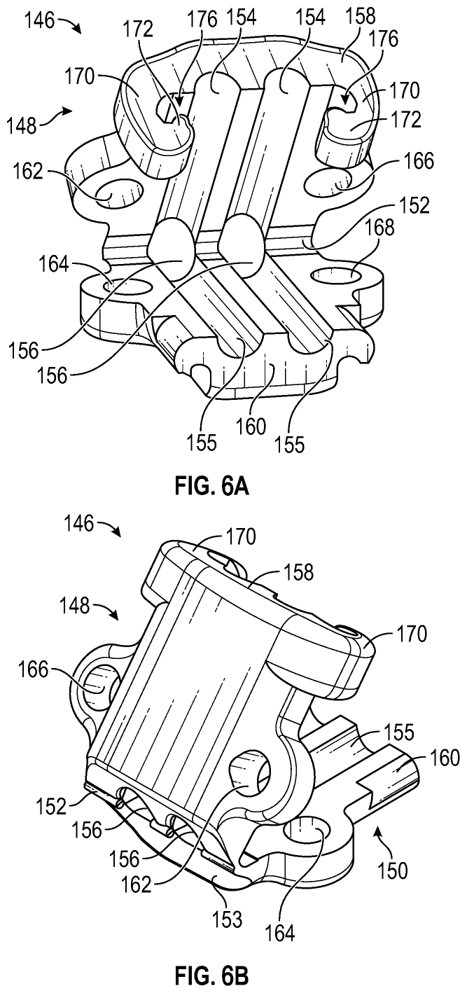

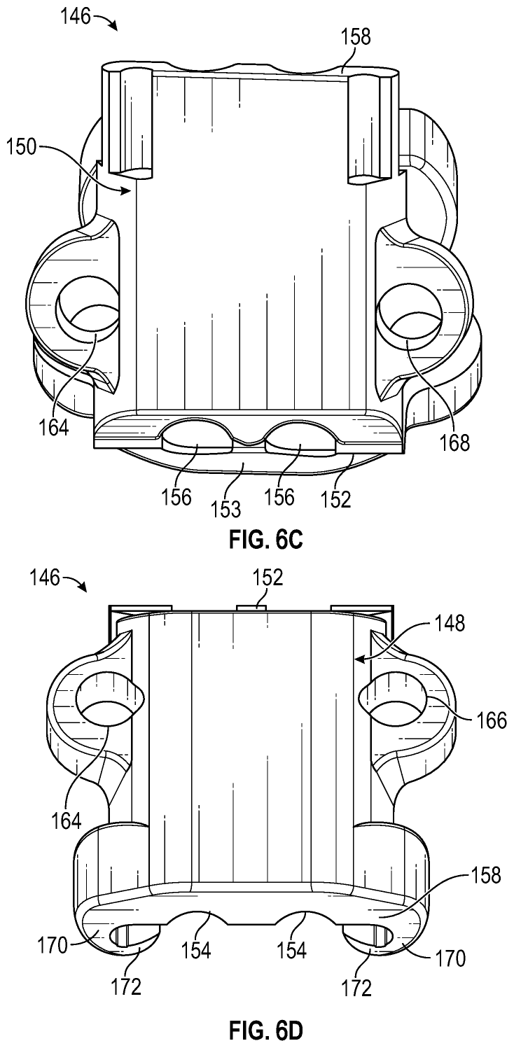

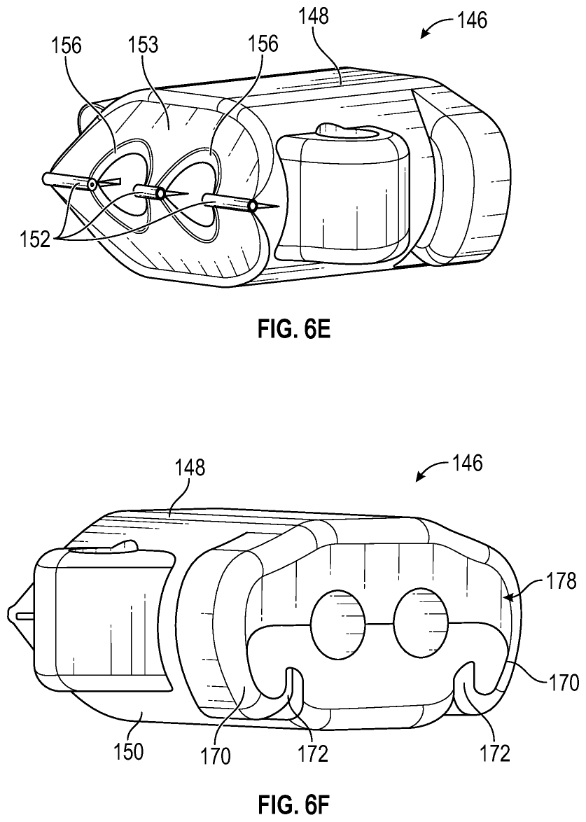

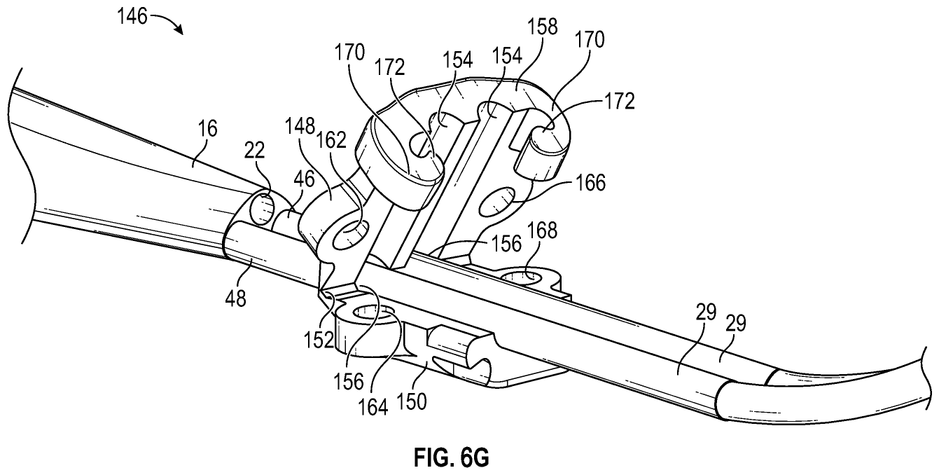

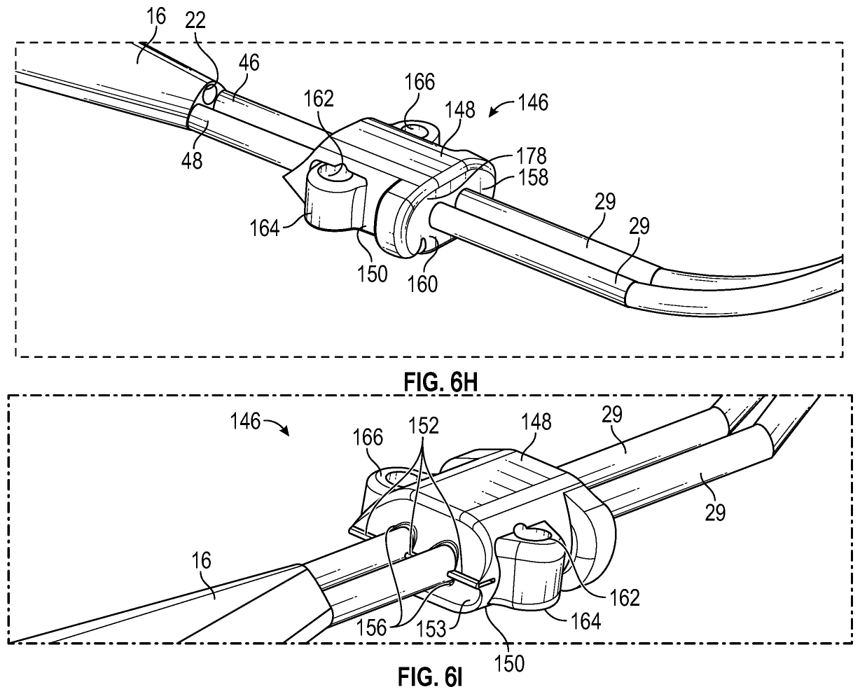

Referring now to FIGS. 6A-6I, a medical anchor 146 is illustrated, according to some embodiments. In some embodiments, the medical anchor 146 may be similar or identical to the medical anchor 78 in terms of one or more features and/or operation.

In some embodiments, the medical anchor 146 may include an upper section 148 and a lower section 150 that fold via a hinge portion 152 disposed between the upper section 148 and the lower section 150. In some embodiments, the upper section 148 and the lower section 150 may include a clamshell shape that moves between an open position and a closed position. In some embodiments, the hinge portion 152 may extend outwardly from a distal face 153 of the medical anchor 146 and/or may be constructed of a flexible material configured to bend.

In some embodiments, an inner surface of the upper section 148 may include one or more grooves 154 configured to align with one or more other grooves 155 of an inner surface of the lower section 150. In some embodiments, when the medical anchor 146 is in the closed position, the electrical conductors 29 may be pinched or clamped within the grooves 154 and the other grooves 155.

In some embodiments, the medical anchor 146 may include one or more openings 156, which may be disposed between the upper section 148 and the lower section 150 and/or extend through the hinge portion 152. In some embodiments, the grooves 152 may extend from a proximal end 158 of the upper section 148 to the openings 156. In some embodiments, the other grooves 154 may extend from a proximal end 160 of the lower section 150 to the openings 156.

In some embodiments, instead of being disposed at a distal end as illustrated in FIG. 6B, the hinge portion 152 may be disposed on a side of the medical anchor 146 between the upper section 148 and the lower section 150. In further detail, in some embodiments, the hinge portion 152 may extend generally parallel to the grooves 155 and/or generally perpendicular to the distal face 153. In some embodiments, the hinge portion 152 may be generally parallel to a longitudinal axis of the medical anchor 146. In these and other embodiments, the openings 156 may not be positioned within the hinge portion 152 but may be formed in response to movement of the medical anchor 146 into the closed position. In these and other embodiments, the electrical conductors 29 may be laid in the other grooves 155 such that when the medical anchor 146 is in the closed position, the electrical conductors 29 may be pinched or clamped within the grooves 154 and the other grooves 155.

In some embodiments, the upper section 148 may include a first suture hole 162 configured to align with a first suture hole 164 of the lower section 150 when the medical anchor 146 is in the closed position. In some embodiments, the upper section 148 may include a second suture hole 166 configured to align with a second suture hole 168 of the lower section 150 when the medical anchor 146 is in the closed position. In some embodiments, suture may be inserted through one or more of the first suture hole 162, the first suture hole 164, the second suture hole 166, and the second suture hole 168 and secured to tissue of the patient to prevent migration of the implantable medical lead 10 after implantation. In some embodiments, the first suture hole 162 may be disposed on an opposite side of the upper section 148 from the second suture hole 166 and/or the first suture hole 164 may be disposed on an opposite side of the lower section 150 from the second suture hole 168.

In some embodiments, the upper section 148 may be configured to snap to the lower section 150 to secure the medical anchor 146 in the closed position. In further detail, in some embodiments, the upper section 148 may include two arms 170, which may oppose each other. In some embodiments, the two arms 170 may extend downwardly from the upper section 148 and/or may include curved ends 172 configured to snap onto a flange 174 of the lower section 150. In some embodiments, a shape of the flange 174 may be a same shape as a shape of a space 176 between the curved ends 172 and the inner surface of the upper section 148. Thus, in some embodiments, when the medical anchor 146 is in the closed position, fluid may not enter through the space 176. In some embodiments, a proximal face 178 of the medical anchor 146 may be smooth, which may reduce damage or irritation of tissue of the patient.

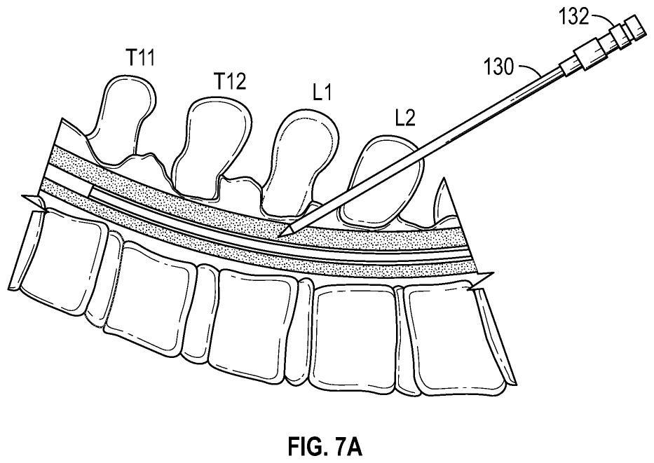

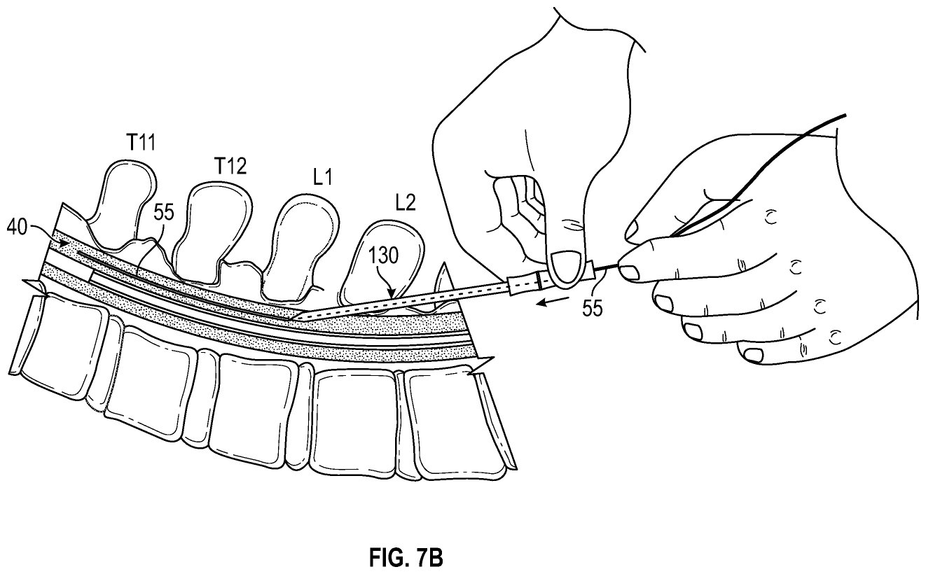

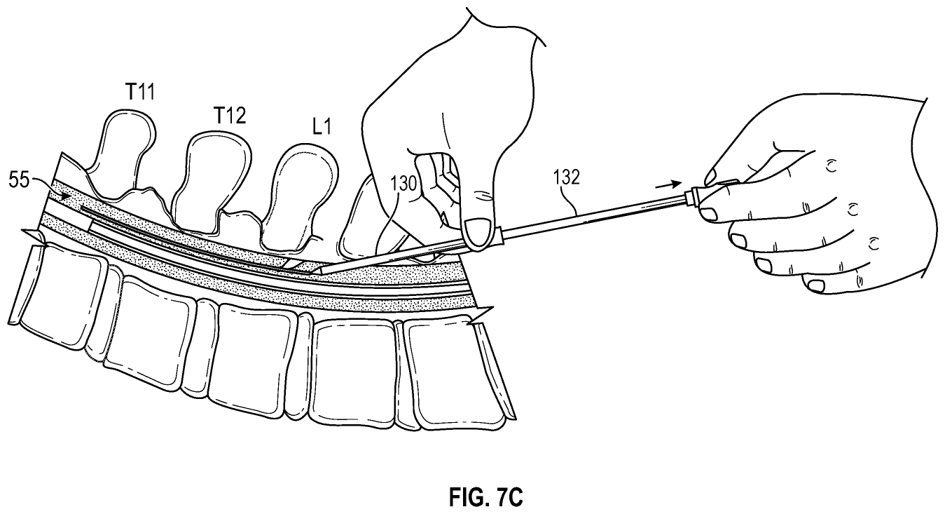

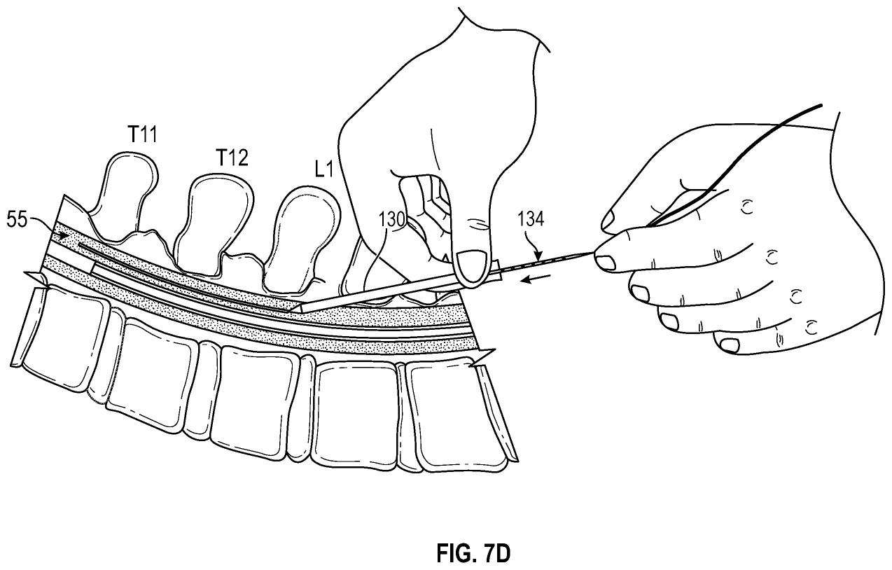

Referring now to FIGS. 7A-7D, a method of using a cannula 130 and the guidewire 55 to implant an implantable medical lead 134 within a patient is illustrated, according to some embodiments. In some embodiments, the cannula 130 and the implantable medical lead 134 may be similar or identical to the cannula 50 and the implantable medical lead 10, respectively, in terms of one or more features and/or operation.

In some embodiments, the cannula 130 may be inserted through the skin of the patient and into the epidural space, as illustrated, for example, in FIG. 7A. In some embodiments, after the cannula 130 is inserted into the epidural space, the guidewire 55 may be inserted through the cannula 130 and into the epidural space beyond a distal end of the cannula 130, as illustrated, for example, in FIG. 7B. In some embodiments, an introducer 132 may be used, and the guidewire 55 may be inserted through the cannula 130 and the introducer 132 into the epidural space beyond the distal end of the cannula 130. In some embodiments, after the guidewire 55 is inserted into the epidural space, the introducer 132 may be withdrawn proximally and removed, as illustrated, for example, in FIG. 7C. In some embodiments, the method may not include use of the introducer 132, and the guidewire 55 may be inserted directly into the cannula 130.

In some embodiments, after the cannula 130 and the guidewire 55 are inserted into the patient at a desired location, the implantable medical lead 134 may be inserted through the cannula 130 into the epidural space. In some embodiments, a surgical instrument, such as the surgical instrument 56 of FIGS. 3A-3F, may be used to insert the implantable medical lead 134 through the cannula 130 and into the epidural space. In other embodiments, the clinician may feed the implantable medical lead 134 through the cannula another suitable driver or his or her hand. In some embodiments, the guidewire 55 may extend through an aperture of the implantable medical lead 134, and the implantable medical lead 134 may move along the guidewire 55 into a desired position proximate spinal nerve tissue to be stimulated. In some embodiments, a position of the implantable medical lead 134 may then be verified using one or more radiopaque markers, such as, for example, the radiopaque markers 42. Although “T11”, “T12” and “L1” are illustrated in FIGS. 7A-7D, it is understood that an insertion point of the implantable medical lead 134 may vary.

In some embodiments, the cannula 130 may be removed after the implantable medical lead 134 is positioned within the epidural space at a desired location. In some embodiments, an anchor, such as, for example, the medical anchor 78 or the medical anchor 146, may then be used to keep the implantable medical lead 134 in position.

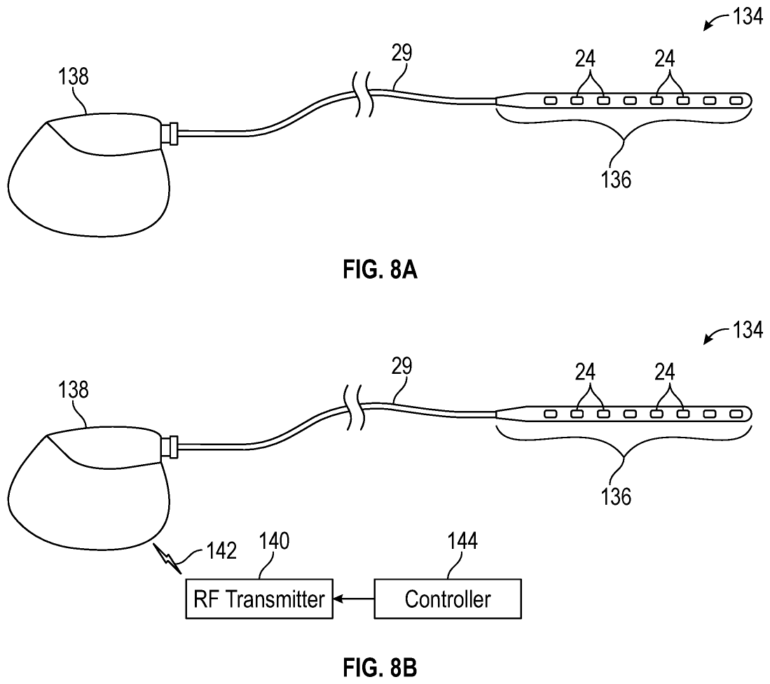

Referring now to FIG. 8A, in some embodiments, after the implantable medical lead 134 is secured to the tissue via the anchor, one or more electrical conductors 29 may then be coupled to an implantable pulse generator (IPG) 136. In some embodiments, a subcutaneous pocket may be created for the IPG 136, and the IPG may be inserted within the subcutaneous pocket.

Referring now to FIG. 8B, in some embodiments, after the implantable medical lead 134 is secured to the tissue via the anchor, one or more electrical conductors 29 may then be coupled to a radiofrequency receiver (RF) 138, which may be operatively coupled to an RF transmitter 140. In some embodiments, the RF transmitter 140 may be operatively coupled to a controller 144 configured to control the RF transmitter 140.

All examples and conditional language recited herein are intended for pedagogical objects to aid the reader in understanding the inventions and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. An implantable medical lead for electrical stimulation, comprising:
a body, comprising:
a flexible distal end;
a flexible proximal end;
a rigid portion extending between the flexible distal end and the flexible proximal end;
an aperture extending through the flexible distal end and the flexible proximal end and configured to receive a guidewire therethrough;
an electrode coupled to the rigid portion; and
an electrical conductor electrically connected to the electrode and extending from the electrode through the flexible proximal end.

2. The implantable medical lead of claim 1, wherein a top surface of the flexible distal end is tapered downwardly in a distal direction.

3. The implantable medical lead of claim 1, wherein a cross-section of the body is dome-shaped or triangular.

4. The implantable medical lead of claim 1, wherein the electrode is disposed on a bottom of the body.

5. The implantable medical lead of claim 1, wherein the body further comprises a plurality of circular radiopaque markers.

6. The implantable medical lead of claim 5, wherein the plurality of circular radiopaque markers comprises a first circular radiopaque marker and a second circular radiopaque marker disposed within the flexible proximal end, wherein the first circular radiopaque marker and the second circular radiopaque marker are equidistant from a longitudinal axis of the body and aligned.

7. The implantable medical lead of claim 1, wherein the body further comprises a plurality of holes, wherein the plurality of holes comprise a plurality of circular radiopaque markers.

8. The implantable medical lead of claim 1, wherein the flexible proximal end is tapered inwardly such that a width of the body decreases in a proximal direction.

9. The implantable medical lead of claim 1, further comprising another electrode coupled to the rigid portion and another electrical conductor electrically connected to the other electrode and extending from the other electrode through the flexible proximal end.

10. The implantable medical lead of claim 9, wherein the flexible proximal end comprises a first opening and a second opening, wherein the electrical conductor extends through the first opening and the other electrical conductor extends through the second opening, wherein the aperture is disposed above and in between the first opening and the second opening.

11. The implantable medical lead of claim 10, further comprising a first plurality of electrodes arranged in a first line and a second plurality of electrodes arranged in a second line parallel to the first line, wherein the first plurality of electrodes comprises the electrode and the second plurality of electrodes comprises the other electrode, wherein the electrical conductor is electrically connected to the first plurality of electrodes, wherein the other electrical conductor is electrically connected to the second plurality of electrodes.

12. The implantable medical lead of claim 1, wherein a bottom surface of the body is flat.

13. The implantable medical lead of claim 1, further comprising a feature compatible for surgical placement via a limited laminotomy performed via an procedure.

14. A medical anchor to secure an implantable medical lead, the medical anchor comprising:
a body, comprising a distal end, a proximal end, an opening extending through the distal end, a groove extending through the proximal end and proximate the opening, a first suture hole opposing a second suture hole, wherein the opening and the groove are configured to receive an electrical conductor; and
a clamp element hinged to the body and configured to move between an open position and a closed position, wherein an inner surface of clamp element comprises another groove configured to align with the groove when the clamp element is in the closed position, wherein the clamp element is configured to clamp the electrical conductor between the clamp element and the body when the clamp element is in the closed position to prevent the electrical conductor from sliding through the medical anchor.

15. The medical anchor of claim 14, wherein the first suture hole and the second suture hole are disposed within the distal end and extend through a top surface of the body and a bottom surface of the body.

16. The medical anchor of claim 14, wherein the body further comprises a cutout portion extending between a first wall and a second wall, wherein the first wall and the second wall are configured to contact a first side and a second side, respectively, of the clamp element when the clamp element is in the closed position, wherein the first wall and the second wall are spaced apart by a floor of the body, wherein the groove extends through the floor.

17. The medical anchor of claim 16, wherein the inner surface of the clamp element is configured to contact the floor when the clamp element is in the closed position.

18. A surgical instrument to implant an implantable medical lead for electrical stimulation, the surgical instrument comprising:
a first arm, comprising:
a first distal end, wherein the first distal end comprises a first clamp surface, wherein the first clamp surface comprises a circular peg configured to insert into a circular hole of an implantable medical lead; and
an aperture extending through the first arm proximal to the first distal end, wherein the aperture is configured to receive a plurality of electrical conductors and a guidewire therethrough; and
a second arm coupled to the first arm, the second arm comprising a second distal end, wherein the second distal end comprises a second clamp surface opposing the first clamp surface, wherein the second clamp surface comprises another circular peg configured to insert into another circular hole of the implantable medical lead.

19. The surgical instrument of claim 18, wherein the second arm comprises a groove opposing the aperture.

20. The surgical instrument of claim 18, wherein the first clamp surface comprises a first rounded indent, wherein the second clamp surface comprises a second rounded indent, wherein the first peg and the second peg extend from the first rounded indent and the second rounded indent, respectively.

21. The surgical instrument of claim 18, wherein the surgical instrument is scissor-tong shaped.
